(19) United States
(12) Patent Application Publication
(10) Pub. No.: US 2002/0010465 A1
(43) Pub. Date: Jan. 24, 2002

(54) Frame fixator and operation system thereof

(75) Inventors: Ja Kyo Koo, Seoul (KR); Jung Soo Han, Seoul (KR); Chang Soo Han, Seoul (KR); In Ho Choi, Seoul (KR); Hyung Joon Sim, Seoul (KR); Bum Seok Park, Seoul (KR); Jung Sung Kim, Seoul (KR); Byung Soo Kim, Seoul (KR); Kyung Tae Kim, Seoul (KR); Chan Soo Shin, Seoul (KR); In Hyuk Cha, Seoul (KR)

Correspondence Address: Merchant & Gould P.C., P.O. Box 2903, Minneapolis, MN, 55402-0903, US

(73) Assignee: Ja Kyo KOO

(21) Appl. No.: 09/771,728

(22) Filed: Jan. 29, 2001

(30) Foreign Application Priority Data
Jan. 31, 2000 (KR) 2000-4646

(51) Int. Cl. A61B 17/66

(52) U.S. Cl. 606/57

(57) ABSTRACT

A frame fixator and operation system thereof, the fixator comprising an upper end ring and a lower end ring, and a plurality of length adjustment means coupled at both ends thereof to the upper end ring and the lower end ring, wherein the length adjustment means include an actuator, a moving member movable by the actuator and a digital indicator for indicating a length of the length adjustment means changeable to the movement of the moving member, the fixator and peration system for enabling to automatically adjust a distraction rate and distraction frequency of fracture during bone deformity correction and lengthening and to easily adjust the length by digitally indicating changed value of length according to manual or automatic manipulation.

### BACKGROUND OF THE INVENTION

1. Field of the Invention

The present invention relates to a hexapod ring type frame fixator used in fixation of fracture and bone lengthening, and more particularly to a frame fixator and operation system thereof adapted for use in automatic adjustment of distraction rate and distraction frequency of fractured piece during correction of bone deformity and bone lengthening.

2. Description of the Prior Art

In general, representative frame fixator for orthopedics used for deformity correction of fracture, bone lengthening, bone and soft tissue include EBI method, Hoffmann method, Mono method, Ilizarov method and the like.

Among these methods of fixators, the frame fixator by Ilizarov method has shown an excellent remedial effect in that therapentic period can be shortened according to accelerated formation of blood cells at fractures and bone, and a stable fixation effect can be obtained according to high resistance to external force such as rotational strain and the like.

The frame fixator by way of Ilizarov method has also demonstrated a great result in fixation of various deformations which might occur after operation.

However, there is a problem in the frame fixator by way of Ilizarov method thus described in that devices should be assembled separately according to shapes of fractures, resulting in requirement of tremendous effort in surgical operation despite excellence in surgical result in bone deformity correction and bone lengthening, and raising a stability problem due to structure complexity.

There is another problem in that a surgical doctor should adjust distraction frequency (surgical operation frequency) and distraction rate (distracted length) at approximately every six hours by manually manipulating devices, such that it is inconvenient for both the doctors and patients to in terms of surgical efficiency and conveniency and accuracy drops due to occurrence of errors resultant from manual adjustment.

### SUMMARY OF THE INVENTION

The present invention is disclosed to solve the aforementioned problems and it is an object of the present invention to provide a frame fixator and operation system thereof arranged to apply a hexapod parallel mechanism used in robotics or machine tools having a function as a general fracture and deformity therapentic device applicable regardless of types fracture and deformity.

It is another object of the present invention to provide a frame fixator and operation system thereof arranged to apply a motor-driven mechanism of adjustable strut, thereby adjusting distraction rate and distraction frequency of fractured pieces automatically during bone deformity correction and bone lengthening.

It is still another object of the present invention to provide a frame fixator and operation system thereof adapted to attach a liquid crystal display (digital indicator) on a strut, allowing a changed valve of strut length according to manual or automatic control to be digitally indicated for easy adjustment of length.

It is further object of the present invention to provide a frame fixator and operation system thereof adapted to use a motor-driven microprocessor to further increase a distraction frequency in comparison with manual manipulation, thereby promoting bone regeneration and enabling a gradual distraction, such that soft tissue damage can be minimized around bones and pains belt by patient can be decreased during bone lengthening to thereby reduce usage of additional drugs such as analgesia and the like.

It is still further object of the present invention to provide a frame fixator and operation system thereof adapted to transmit driving information between a motor-driven system and a computer through an external interface, enabling accurate and simple adjustment even only with manipulation of computer.

In accordance with one object of the present invention, there is provided a frame fixator, the fixator comprising:

an upper end ring and a lower end ring; and

a plurality of length adjustment means coupled at both ends thereof to the upper end ring and the lower end ring, wherein the length adjustment means include an actuator, a moving member movable by the actuator and a digital indicator for indicating a length of the length adjustment means changeable to the movement of the moving member, and the length adjustment means may be any device such as strut utilizing a rod formed with threads, rack, pinion, lead screw and the like for changing a rotary movement to a linear movement, or hydraulic/pneumatic cylinder or linear motor performing a linear movement.

In accordance with another object of the present invention, there is provided an operation system of frame fixator, the frame fixator including an upper end ring, a lower end ring and a plurality of length adjustment means coupled at both ends thereof to the upper end ring and the lower end ring and changeable at length thereof according to movement of moving member, wherein the length adjustment means are operated by a motor driving apparatus for automatically adjusting distraction frequency and distraction rate.

The motor driving apparatus includes control means for outputting a control signal for adjusting distraction frequency and distraction rate of the length adjustment means, a motor driven by the control signal from the control means, a transmitting means for moving the moving member according to operation of the motor, and length measuring means for measuring the length of the length adjustment means according to moving position of the moving member.

The transmitting member may be a gear element such as spur gear, helical gear, worm gear or the like, and an electric device such as spracket chain, belt or the like as well. The length measuring means may use such length measuring device as potentiometer, encoder or the like.

Furthermore, the operation system of the frame fixator according to the present invention further comprises:

a computer for calculating a distraction frequency and a distraction rate of the length adjustment means according to intrinsic program to transmit same to the motor driving apparatus when clinical data such as frame parameter, deformity parameter, mounting parameter and the like are input according to fracture and deformity status of a patient, and interface means for updating data to the frame fixator periodically and unperiodically by data communication and receiving data utilizing the computer.

### BRIEF DESCRIPTION OF THE DRAWINGS

For fuller understanding of the nature and objects of the invention, reference should be made to the following detailed description taken in conjunction with the accompanying drawing in which:

FIG. 1 is an external view of a frame fixator according to the present invention;

FIG. 2 is an external view of a strut applied to the present invention;

FIG. 3 is an inner structural view of actuator mounted with a motor-driving mechanism according to the present invention;

FIG. 4 is a sectional view taken along line A-A′ of FIG. 3;

FIG. 5 is a block diagram of driving system for controlling a motor-driven mechanism at a frame fixator according to the present invention;

FIG. 6 is an initial screen of a computer program for calculating a length adjustment schedule of strut applied to the present invention;

FIG. 7 is a table of length adjustment schedule of a strut output by a computer program for calculating a length adjustment schedule of strut applied to the present invention;

FIG. 8 is a X-ray shooting view for explaining a deformity parameter applied to the present invention;

FIG. 9 is a X-ray view for describing a mounting parameter applied to the present invention;

FIG. 10 is a drawing for structurally describing a process where length adjustment schedule of strut is calculated according to the present invention; and

FIG. 11 is a X-ray shooting view for explaining a residual deformity correction method according to the present invention.

### DETAILED DESCRIPTION OF THE INVENTION

Now, preferred embodiments of the present invention will be described in detail with reference to the accompanying drawings.

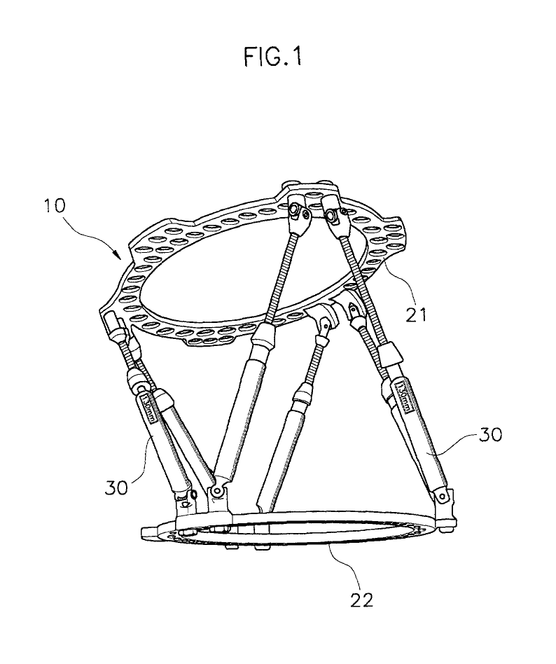

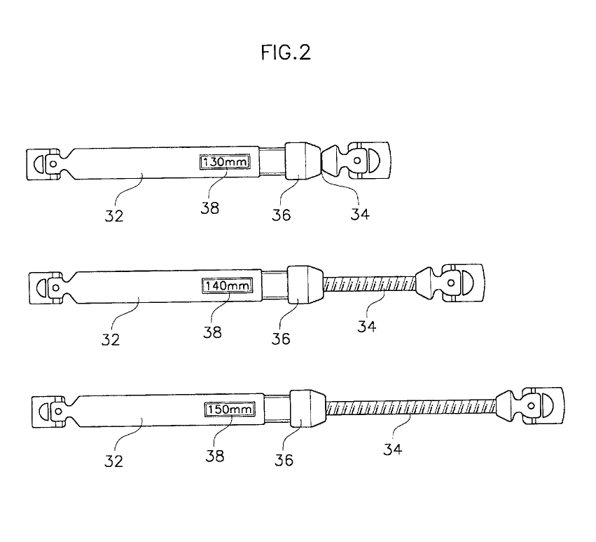

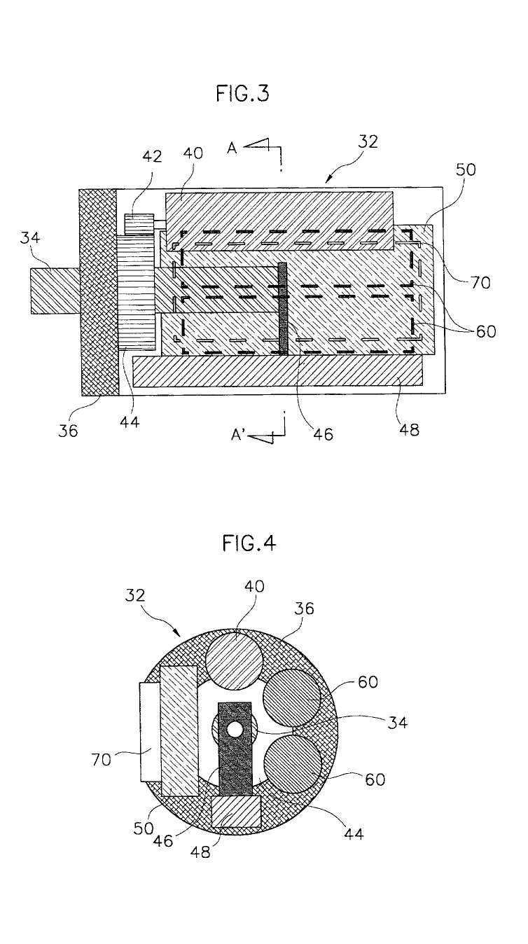

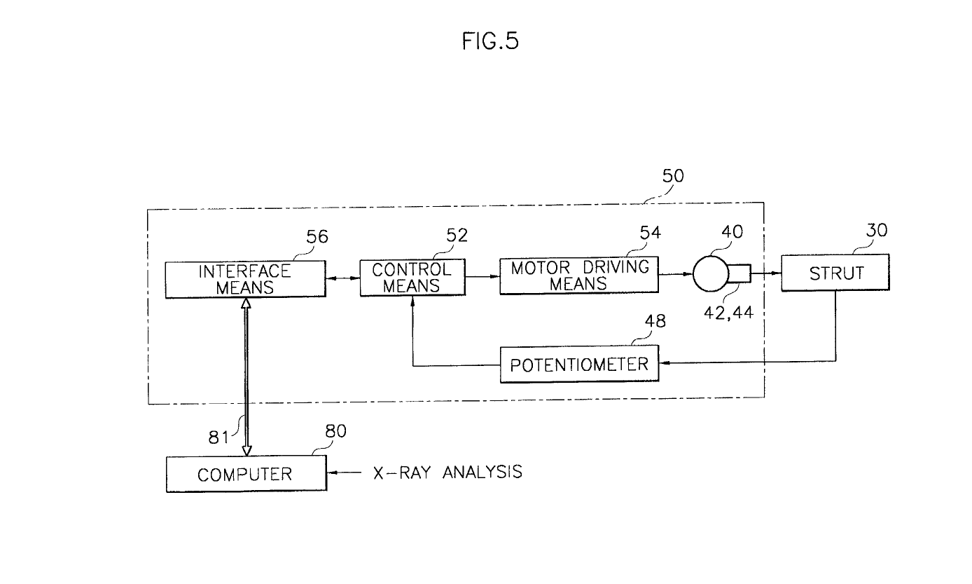

FIG. 1 is an external view of a frame fixator according to the present invention, FIG. 2 is an external view of a strut applied to the present invention, FIG. 3 is an inner structural view of actuator mounted with a motor-driving mechanism according to the present invention, and FIG. 4 is a sectional view taken along line A-A′ of FIG. 3.

As illustrated in FIG. 1, a frame fixator 10 is mounted at an upper side and a lower side thereof with an upper end ring 21 and a lower end ring 22 respectively.

Between the upper end ring 21 and the lower end ring 22, there is disposed a plurality of struts 30 (by way of example, six struts). The plurality of struts 30 are hinged to the upper end ring 21 and the lower end ring 22 by way of universal joint, spherical joint or the like.

As shown in FIG. 2, each strut 30 is comprises of an actuator 32, and a rod 34 coupled to the actuator 32 via a nut 36 and formed with threads for moving along according to rotating manipulation of the nut 36.

The actuator 32 is formed at a peripheral side thereof with a liquid crystal display (LCD) 38 for digitally indicating a total length (mm) of the strut 30 changing to the movement of the rod 34 formed with threads and for indicating an operation mode (manual and automatic), power status (voltage) and computer connection status.

As shown in FIGS. 3 and 4, the actuator 32 in installed therein with a motor 40 for generating a driving force in order to move the rod 34 formed with the threads. The motor 40 is disposed at an axle thereof with a first gear 42 while the first gear 42 is coupled at one side thereof via a nut 36 to a second gear 44 for moving the rod 34 formed with the threads by being meshed with the first gear 42.

Furthermore, the thread-formed rod 34 is disposed at one tip end thereof with a guide member 46 for move in cooperation with the movement of the rod 34 formed with the threads. The guide member 46 is provided at one side thereof with a potentiometer 48 for utilizing voltage value or resistance value changing to the movement of the guide member 46 to thereby measure the length of the strut 30.

The actuator 32 includes a motor driving apparatus 50 for driving the motor 40 to automatically adjust the length of the strut 30 according to length adjustment schedule of the strut 30 input from a computer 80 and stored therein, and power means 60 for supplying power to the motor driving apparatus 50 and the motor 40. The motor driving apparatus 50 is mounted at one side thereof with a LCD driving unit 70 for driving the LCD 38 according to a control signal from the motor driving apparatus 50.

The power means 60 is a battery or a device for supplying power by way of external input, where the battery may be a common disposable battery or a battery rechargeable by an external direct current (DC) supply device.

Next, operation system for controlling a motor-driven mechanism of a frame fixator thus constructed will be described with reference to FIG. 5.

As shown in FIG. 5, the operation system of the frame fixator includes the motor-driving apparatus 50 for driving the motor 40 according to a length adjustment schedule of strut 30 stored by input from the computer 80 to automatically adjust the length of the strut 30, and the computer 80 for calculating a length adjustment schedule of strut (length adjustment period of strut and length of strut at this period) according to fractured state and deformity state of a patient to transmit same to the motor driving apparatus 50 via a transmission cable 81, where the transmission cable 81 may be connected to or disconnected from the motor driving apparatus 50 according to the need of a surgical doctor.

The motor driving apparatus 50 includes control means 52 for automatically adjusting the length of the strut 30 according to the length adjustment schedule stored therein by input from the computer 80, motor driving means 54 for driving the motor 40 according to a control signal output from the control means 52, gears 42 and 44 for moving the rod 34 formed with threads according to the drive of the motor 40, a potentiometer 48 for measuring a length of the strut 30 according to movement position of the rod 34 formed with the threads to input same to the control means 52, and interface means 56 for updating the length adjustment schedule of the strut periodically and unperiodically according to manipulation of the surgical doctor.

The control means 52 is a central processing unit internally comprising a timer, a control device, a control memory device, a resistor, an anithmetic and logic unit (ALU), an interface and the like. The control means 52 is usually made of a circuit board but in case of a small processor, a microprocessor chip comprising a very large scale interface (VLSI) performs the function of the central processing unit.

The interface means 56 is an apparatus for data communication and data receipt utilizing the computer 80. In other words, the apparatus serves to connect a common bordering portion when two systems or apparatuses such as the motor driving apparatus 50 and the computer 80 are connected, where IEEE-488, RS-232C and the like are used.

When a surgeon understands or grasps fractured state or deformity state of a patient according to X-ray film to compare same with a normal state and inputs clinical data such as initial input value of three (3) frame parameters, six (6) deformity parameters showing the degree of deformity, four (4) mounting parameters illustrating bone position relative to center of upper end ring 21 or lower end ring 22, the computer 80 performs a kinematic analysis based on the clinical data thus input to calculate a length adjustment schedule of strut, whereby soft tissue or nerve system around bone may not be damaged and the frame fixator 10 may have an adequate trace, move and reach a final position, because a program is inherently stored therein for automatically calculating the length adjustment schedule of strut.

Now, operational effect of the frame fixator thus constructed and operation system thereof will e described.

A surgeon attaches a frame fixator to a patient for treatment of fracture or correction of chronic deformity.

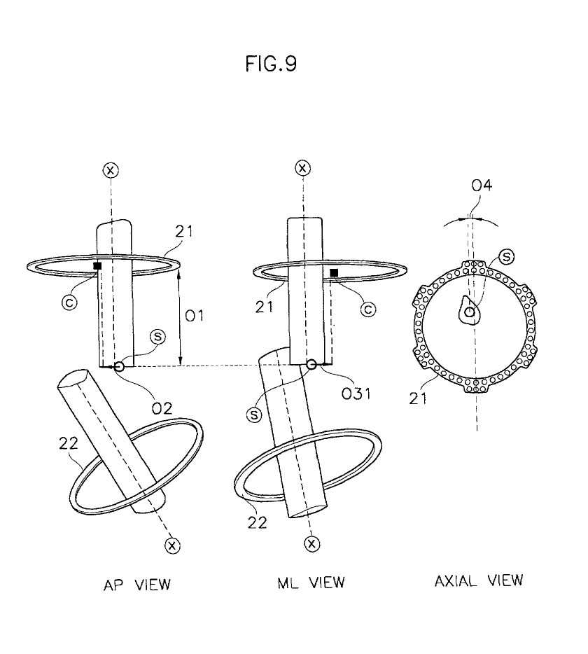

First, fracture from accident or chronic deformity state is grasped according to X-ray film (front and lateral view) of a patient and the upper end ring 21 and the lower end ring 22 are adequately selected according to internal diameters thereof by vertical fixation into a fracture part as illustrated in FIG. 9.

At this time, the upper end ring 21 and the lower end ring 22 are not made parallel because these two rings 21 and 22 are respectively and vertically secured by a wire relative to lengthwise axle □ of each fractured piece.

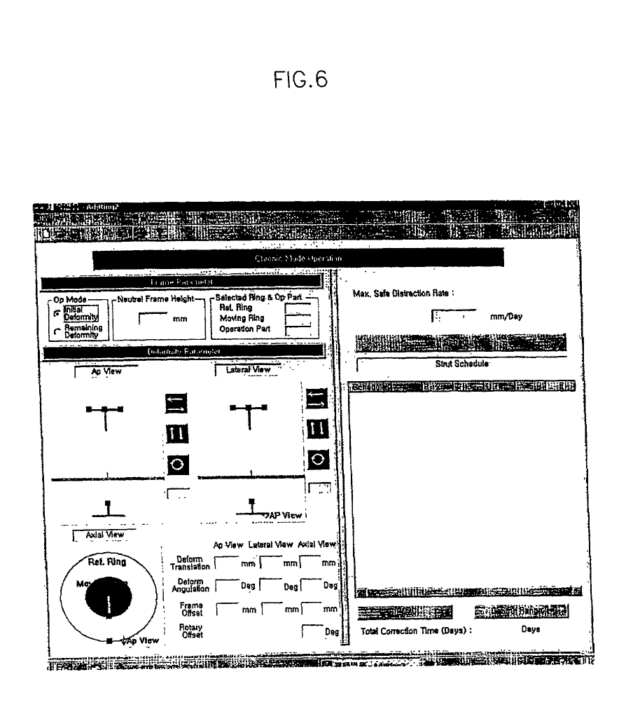

Successively, a deformity correction mode (initial deformity correction mode or remaining deformity correction mode) and operation site (by way of example, femur or fibula) is selected from screen of the computer 80 in FIG. 6, where, three (3) frame parameters, six (6) deformity parameters and four (4) mounting parameters are respectively input by way of a mouth or a keyboard to thereafter input maximum safe distraction rate.

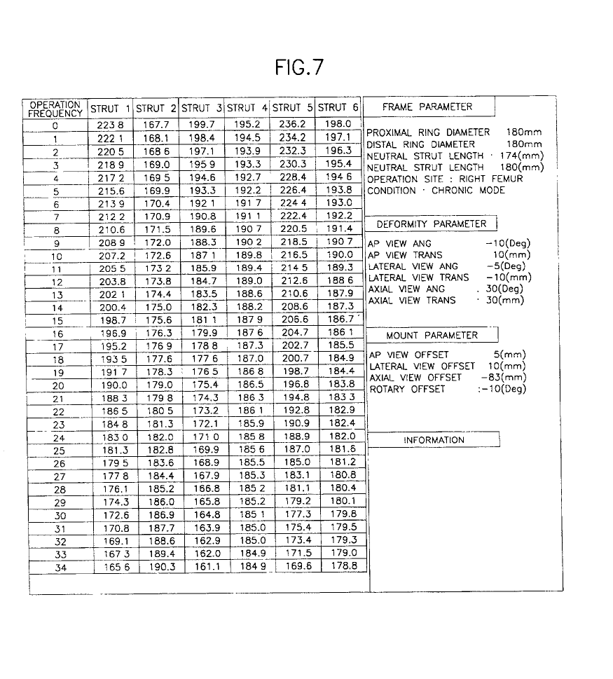

When a schedule button on the screen of the computer 80 in FIG. 6 is clicked to perform simulation, length adjustment schedule of each strut (length adjustment period of strut and schedule relative to the length of strut at this period) is calculated for display such that the program stored in the computer 80 gradually adjusts the length of the strut 30 to allow the upper end ring 21 and the lower end ring 22 to gradually move from fracture state of state depicting the deformity (a state before initial deformity correction) to neutral state (a state after initial deformity correction). Therefore no simulation is needed to be made at each operation by outputting in one document contents on screen as illustrated in FIG. 7, such that documents already output can be utilized. At this time, length adjustment schedule of each strut is calculated lest distraction rate of fractured piece should exceed the maximum safety distraction rate.

When the length adjustment schedule of strut is calculated as per the foregoing explanation, the surgeon manually adjusts the lengths of six (6) (S1-S6) struts to initial strut lengths on the length adjustment schedule of the strut (by way of example, S1=223.8, S2=167.7, S3=199.7, S4=195.2, S5=236.2 and S6=198.0).

When the surgeon turns the nut 36 coupled to the actuator 32 of the strut 30 in order to manually adjust the length of each strut 30, the rod 34 formed with threads meshed to actuator 32 via the nut 36 linearly moves according to rotated manipulation of the nut 36 to cooperatively move the guide member 46 coupled to a tip end of the rod 34 formed with treads.

Successively, the potentiometer 48 disposed at one side of the guide member 46 measures the length of the strut according to moved position of the rod 34 formed with threads to digitally indicate same on the LCD 38, such that the surgeon can easily, accurately and manually adjust the length of the strut 30. The six (6) struts (S1-S6) adjusted by way of initial strut lengths according to the length adjustment schedule are hinged to the upper end ring 21 and the lower end ring 22 secured to the patient via universal joint, spherical joint or the like to complete installment of the frame fixator 10 as illustrated in FIG. 1.

Furthermore, with the completion of installment of the frame fixator 10, actuator 32 mounted to each strut 30 is connected to the computer 80 through transmission cable 81, whereby length adjustment schedule of each strut calculated from the computer 80 is input to the control means for storage via interface means 56 of the motor driving apparatus mounted at each actuator 32.

Successively, the control means 52 of the motor driving apparatus 50 outputs to the motor driving means 54 a control signal for adjusting the length of the strut 30 according to the length adjustment schedule of strut input and stored through the interface means 56.

The motor driving means 54 receives the control signal output from the control means 52 to drive the motor 40 and when the motor is driven, a first gear 42 coupled to an axle of the motor 40 is rotated and a second gear 44 meshed with the first gear 42 is rotated to move the rod 34 formed with threads coupled to the nut 36.

When the rod 34 formed with the threads is moved, the guide member 46 coupled to a tip end of one side of the rod 34 formed with the threads is simultaneously moved while the potentiometer 48 measures the length of the strut 30 according to moved position of the rod 34 formed with the threads to input same to the control means 52.

The control means 52 compares the length of the strut 30 measured by the potentiometer 48 with the length of the strut on the stored length adjustment schedule to gradually adjust the strength of the strut 30 to a neutral position for treatment of fracture or chronic deformity.

Conclusively, the strength of the strut 30 at the frame fixator 10 is automatically adjusted abruptly or gradually (distraction rate and distraction frequency of fractured piece are automatically adjusted) according to the length adjustment schedule of strut calculated by the computer 80, and when the upper end ring 21 and the lower end ring 22 are paralled, the fractured pieces are aligned on a straight line to complete an initial deformity correction.

Meanwhile, process of calculating length adjustment schedule of each strut from the computer 80 will be further described in detail.

The three (3) frame parameters comprise diameters of the upper end ring 21 and the lower end ring 22 and neutral frame height, where the neutral frame height is a distance between the upper end ring 21 and the lower end ring 22 obtained following the initial deformity correction. The length of the neutral strut may be used instead of the neutral frame height.

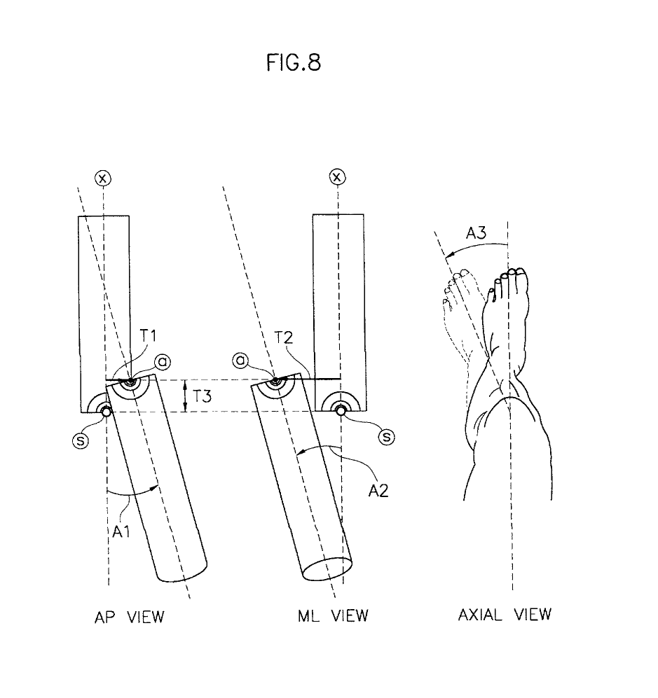

The six (6) deformity parameters represent values of deformity degrees, and, as illustrated in FIG. 8, include angulation (A1), translation (T1) at Anterior-Posterior view (AP view), angulation (A2) and translation (T2) at Medial-Lateral view (ML view), and angulation (A3) and translation (T3) at Axial view, when the surgeon takes a corresponding point prior to operation and an origin point after the operation according to the X-ray film (AP view and ML view).

At this time, the deformity parameter holds a different meaning each at an initial deformity correction mode and at a residual, that is, the initial deformity correction mode defines a start value while the residual correction mode represents a target value.

Furthermore, the four (4) mounting parameters indicate a bone position relative to a centerline from reference ring (the lower end ring in case of Femur and the upper end ring for fibula) on AP or Lateral films, and as depicted in FIG. 9, comprise an Axial view offset (01) from a normal reference point □ following operation, AP view offset and ML view offset (02, 03) of a normal reference point □ after surgery from a center point © of the reference ring at front side and lateral side and Rotary offset (starting point according to serial number) (04) from the center point © of the reference ring.

Successively, when the thirteen (13) clinical data including the three (3) frame parameters, six (6) deformity parameters and four (4) mounting parameters are input to the computer 80 and simulation is performed following input of the maximum safet distraction rate, programs stored in the computer 80 calculates length adjustment schedule (length adjustment timing and schedule relative to strut length at the timing) of each strut by way of analysis of mechanism.

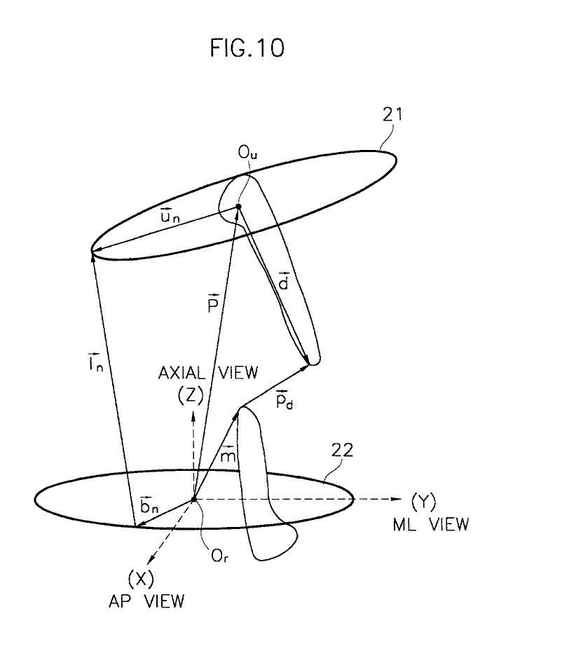

FIG. 10 is an example of femur operation, where the lower end ring 22 becomes a reference ring while the upper end ring 21 functions as a moving ring.

Furthermore, directions of AP view, ML view and Axial view are respectively represented along X, Y, Z coordinate axes, whereas a center point of the moving ring is given as “Ou” and a center point of reference ring is defined as “Or”. And an origin point is arbitrarily determined with reference to a point where two detached bones join.

A calculating method by way of kinetics of machinery for calculating each strut length (Iₙ) gradually adjusted in FIG. 10 is given as below in Formula 1.

$${l}_{n} = \sqrt{{( \vec{P} + R \vec{{u}_{N}} - \vec{{b}_{n}} )}^{T} ( \vec{P} + R \vec{{u}_{n}} - \vec{{b}_{n}} )} \qquad \text{Formula 1}$$

where, \vec{P} is a potential vector between a center of reference ring and a center of moving ring, and may be calculated by Formula 2.

Furthermore, R is a rotary matrix between the reference ring and the moving ring, and may be obtained by Formula 6. \vec{uₙ} is a potential vector (n=1 . . . 6) of each joint with reference to center of the moving ring, while \vec{bₙ} is a potential vector (n=1 . . . 6) of each joint relative to center of reference ring.

$$\vec{P} = \vec{m} + \vec{{P}_{d}} - R \vec{d} \qquad \text{Formula 2}$$

where, \vec{m} is a potential vector of origin point with respect to center of reference ring and may be obtained by Formula 3, \vec{P_{d}} is a potential vector for indicating amount of deformity, and may be calculated by Formula 4, where it is a starting value at initial deformity correction mode and is a target value at residual deformity correction mode. \vec{d} is a potential vector of origin point with reference to center of moving ring and may be calculated by Formula 5.

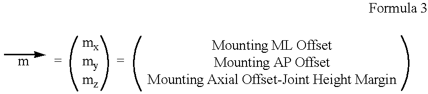

where, a joint height margin is a height of joint equipped at the frame fixator

$$\vec{{P}_{d}} = ( \begin{matrix}{P}_{x} \\ {P}_{y} \\ {P}_{z}\end{matrix} ) \qquad \text{Formula 4}$$

where, Pₓ, P_{y} and P_{z} are translations of AP view, ML view and Axial view respectively.

$$\vec{d} = ( \begin{matrix}{d}_{x} \\ {d}_{v} \\ {d}_{z}\end{matrix} ) = ( \begin{matrix}0 \\ 0 \\ Neutral Frame Height - Mounting Axial offset - Joint Height Margin\end{matrix} ) \qquad \text{Formula 5}$$

$$R = Rot ( {θ}_{up} , χ ) Rot ( {θ}_{lt} , y ) Rot ( {θ}_{ax} + {m}_{θ} , z ) \qquad \text{Formula 6}$$

where, θᵤₚ, θₗₜ and θₐₓ are respectively angulations of AP view, ML view and Axial view while m_{θ} is a rotary frame offset.

At this time, a correction period is determined by a period from an initial deformity correction state (state before surgery) to a final deformity correction state (state after completion of surgery) divided by maximum safe distraction rate (r), and \vec{P_{d}_{ᵢ}}, and Rₜ may be determined by utilizing the following Formula 7 and Formula 8 with respect to correction period thus determined.

$$\begin{matrix}\vec{{P}_{{d}_{i}}} = ( \begin{matrix}{P}_{x} = - i \frac{{P}_{x}}{r} \\ {P}_{y} = - i \frac{{P}_{y}}{r} \\ {P}_{z} = - i \frac{{P}_{z}}{r}\end{matrix} ) & \text{Formula 7} \\ {R}_{i} = Rot ( {θ}_{ap} - i \frac{{θ}_{ap}}{r} ) Rot ( {θ}_{lt} - i θ \frac{{t}_{lt}}{r} ) Rot ( {θ}_{ax} + {m}_{θ} - i \frac{{θ}_{ax}}{r} ) \\ ( i = 0 , 1 , 2 … maximum correction frequency ) & \text{Formula 8}\end{matrix}$$

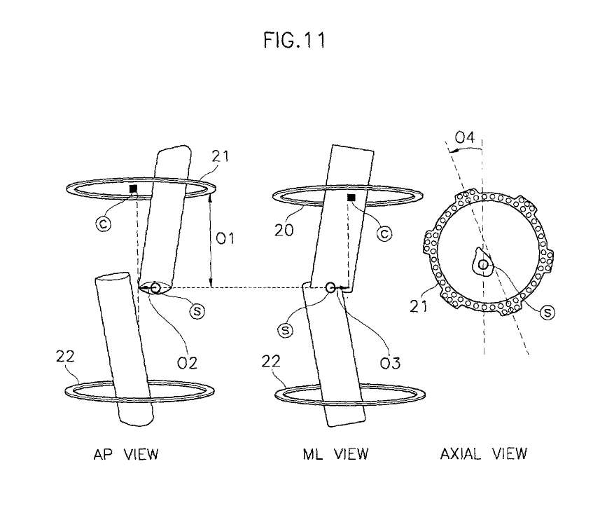

Meanwhile, in case the initial deformity correction is performed as in FIG. 11, and the upper end ring 21 and the lower end ring 22 are paralleled but fractured pieces are not straightened, next step is to enter a final treatment stage which is a remaining deformity correction mode.

In other words, a surgeon inputs the three (3) frame parameters, six (6) deformity parameters and four (4) mounting parameters according to X-ray film of a patient.

When the maximum safe distraction rate is input and simulation is performed, the computer program gradually adjusts the strut length to calculate length adjustment schedule of each strut with reference to six (6) struts 30 such that the upper end ring 21 and the lower end ring 22 each at neutral state can be moved to deformity state where abnormalality state is corrected according to the schedule. The length adjustment schedule of each strut thus described is reverse from schedule where the initial deformity correction mode is output.

At this time, the remaining deformity correction is performed after the initial deformity correction and the three (3) frame parameters and four (4) mounting parameters are the same as before, such that six (6) deformity parameters are only input for use.

As described in the above embodiment, the length adjustment schedule of strut calculated from the computer is re-transmitted to the control means 52 at the motor driving apparatus 50 through the interface means 56, and the motor driving means 54 receives a control signal output from the control means 52 and drives the motor 40 to adjust the parallel-fixed upper end ring 21 and lower end ring 22 at deformed state to correct the remaining deformity state.

At the same time, LCD driving unit 70 digitally indicates the length of strut 30 through the LCD 38 according to the control signal output from the control means 52 to enable the surgeon or the patient to easily check the length of the strut 30 and also indicates operation mode (automatic).

As mentioned above, the six (6) struts (S1-S6) of struts hinged to the upper end ring 21 and the lower end ring 22 at the frame fixator 10 are automatically adjusted according to schedule by the motor driving apparatus 50 mounted inside each actuator 32 to provide a clinical convenience to both the surgeon and patient.

Furthermore, the distraction frequency may be increased at need, and increase of distraction frequency enables the width of distraction rate to be finely adjusted, such that accuracy thereof may be heightened compared with manual manipulation.

While the embodiment of the present invention has been described in terms of the strut 30 utilizing the rod 34 formed with threads for adjustment of distraction rate and distraction frequency, the present invention is not limited to the above description. It should be apparent rack, pinion, lead screw, pneumatic/hydraulic cylinders, linear motor and the like may be used to accomplish the same object and effect as those of the present invention.

Furthermore, while the embodiment of the present invention has been described with reference to a spur gear comprising first and second gears 42 and 44 for movement of rod 34 formed with threads, the present invention is not limited to the above description. It should be apparent that helical gear, worm gear, other forms of gear electrical instruments such as spracket-chain driving, belt driving and the like may be used to accomplish the same object and effect as those of the present invention.

Still furthermore, while the embodiment of the present invention has been described with respect to the potentiometer 48 for measurement of strut 30, the present invention is not limited to the above description. It should be noted that an encoder may be used to accomplish the same object and effect as those of the present invention.

As apparent from the foregoing, there is an advantage in the frame fixator and operation system thereof thus described according to the present invention in that a hexapod ring type frame fixator used for robotics or machine tools is applied to obtain a function as general fracture and deformity treatment instrument applicable regardless of fracture and deformity shape and to apply a motor driving mechanism applicable for strut length adjustment for automatic adjustment of distraction rate and distraction frequency in bone deformity correction and bone lengthening.

There is another advantage in that a strut is attached with an LCD to digitally indicate changed values of strut lengths according to manual or automatic adjustment, enabling an easy length adjustment, and motor driving using a microprocessor further increases compared with manual manipulation to promote bone regeneration in bone tissue, enabling a gradual lengthening to minimize soft tissue damage around bone and to reduce pain felt by a patient in lengthening for reduction of additional drug stuffs such as analgesic or the like.

There is still another advantage in that an external interface transmits a driving information between a motor driving system and a computer to make it possible to simply and accurately adjust only through manipulation of computer, and a driving data in treatment period can be stored in a controller to independently operate the frame fixator, while, even in the case of long time of treatment, data base can be used to systematically control the medical treatment of a patient

What is claimed is:

1. A frame fixator, the fixator comprising:
an upper end ring and a lower end ring; and
a plurality of length adjustment means coupled at both ends thereof to the upper end ring and the lower end ring, wherein the length adjustment means include;
an actuator;
a moving member movable by the actuator; and
a digital indicator for indicating a length of the length adjustment means changeable to the movement of the moving member.

2. An operation system of frame fixator, the frame fixator including an upper end ring, a lower end ring and a plurality of length adjustment means coupled at both ends thereof to the upper end ring and the lower end ring and changeable at length thereof according to movement of moving member, wherein the length adjustment means are operated by a motor driving apparatus for automatically adjusting distraction frequency and distraction rate.

3. The operation system of frame fixator as defined in claim 2, wherein the motor driving apparatus comprises:
control means for outputting a control signal for adjusting distraction frequency and distraction rate of the length adjustment means;
a motor driven by the control signal from the control means;
a transmitting means for moving the moving member according to operation of the motor; and
length measuring means for measuring the length of the length adjustment means according to moving position of the moving member.

4. The operation system of frame fixator as defined in claim 3, further comprising a computer for calculating a distraction frequency and a distraction rate of the length adjustment means according to intrinsic program to transmit same to the motor driving apparatus when clinical data such as frame parameter, deformity parameter, mounting parameter and the like are input according to fracture and deformity status of a patient.

5. The operation system of frame fixator as defined in claim 4, further comprising interface means for updating data to the frame fixator periodically and unperiodically by data communication and receiving data utilizing the computer.
